(19) United States
(12) Patent Application Publication
(10) Pub. No.: US 2020/0297415 A1
(43) Pub. Date: Sep. 24, 2020

(54) AUTOMATED ELECTRODE RECOMMENDATION FOR ABLATION SYSTEMS

(71) Applicant: Boston Scientific Scimed Inc., Maple Grove, MN (US)

(72) Inventors: Jeffry V. Marshik, New Brighton, MN (US); Jacob I. Laughner, St. Paul, MN (US); Adam J. Reinhardt, St. Paul, MN (US); Michael Sean Coe, Plymouth, MN (US); Christopher Joel Robinson, Coon Rapids, MN (US); Heather Shumaker, Wood, MN (US)

(21) Appl. No.: 16/824,985

(22) Filed: Mar. 20, 2020

Related U.S. Application Data
(60) Provisional application No. 62/822,142, filed on Mar. 22, 2019.

(51) Int. Cl. A61B 18/14; A61B 18/12; A61B 34/00; A61B 90/00; G06N 3/08

(52) U.S. Cl. CPC A61B 18/1492; A61B 18/1206; A61B 2018/00577; A61B 90/361; G06N 3/08; A61B 34/25

(57) ABSTRACT

An ablation system includes a radiofrequency (RF) generator configured to generate RF energy; an ablation catheter in communication with the RF generator and including a plurality of ablation electrodes; a camera positioned on the ablation catheter and arranged to take an image that includes at least some of the plurality of ablation electrodes; and one or more processors configured to recommend a subset of the plurality of ablation electrodes to be activate ablation electrodes based, at least in part, on the image.

### CROSS REFERENCE TO RELATED APPLICATION

This application claims priority to Provisional Application No. 62/822,142, filed Mar. 22, 2019, which is herein incorporated by reference in its entirety.

### TECHNICAL FIELD

The present disclosure relates generally to systems, devices, and methods involving cardiac ablation.

### BACKGROUND

Various cardiac abnormalities can be attributed to improper electrical activity of cardiac tissue. Such improper electrical activity can include generation of electrical signals, conduction of electrical signals of the tissue, etc., in a manner that does not support efficient and/or effective cardiac function. For example, an area of cardiac tissue may become electrically active prematurely or otherwise out of synchrony during the cardiac cycle, causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactive tissue (e.g., scar tissue) may be preferable to malfunctioning cardiac tissue.

### SUMMARY

In Example 1, a computer-implemented method includes recommending, via one or more processors and a graphical user interface, a set of ablation electrodes to activate based, at least in part, on analysis of an image containing the ablation electrodes.

In Example 2, the method of Example 1, wherein the analysis includes recognizing the ablation electrodes in the image.

In Example 3, the method of any of Examples 1 or 2, wherein the analysis includes comparing contrast of pixels in the image around the ablation electrodes.

In Example 4, the method of any of Examples 1-3, wherein the analysis determines which of the ablation electrodes are selectable.

In Example 5, the method of any of Examples 1-4, wherein the analysis is carried out by a trained neural network.

In Example 6, the method of any of Examples 1-5, wherein the recommending is also based, at least in part, on impedance measurements.

In Example 7, the method of Example 6, wherein the impedance measurements determine which of the ablation electrodes are selectable.

In Example 8, the method of Example 7, wherein the selectable ablation electrodes are ablation electrodes associated with an impedance measurement within a predetermined range of impedance values.

In Example 9, the method of any of Examples 4, 5, 7, and 8, further comprising assigning, via the one or more processors, each of the recommended ablation electrodes to be either a sink or a source.

In Example 10, the method of any of Examples 1-9, wherein the recommending is based, at least in part, on balancing estimated power received by source electrodes.

In Example 11, the method of any of Examples 1-10, wherein the recommending is based, at least in part, on limiting estimated power received by source electrodes below a predetermined threshold.

In Example 12, the method of any of Examples 1-11, wherein the recommended set of ablation electrodes forms a closed circuit.

In Example 13, a computing device adapted to execute the steps of the method of Examples 1-12.

In Example 14, a computer program product comprising instructions to cause the one or more processors to carry out the steps of the method of Examples 1-12.

In Example 15, a computer-readable medium having stored thereon the computer program product of Example 14.

In Example 16, an ablation system includes a radiofrequency (RF) generator configured to generate RF energy; an ablation catheter in communication with the RF generator and including a plurality of ablation electrodes; a camera positioned on the ablation catheter and arranged to take an image that includes at least some of the plurality of ablation electrodes; and one or more processors configured to recommend a subset of the plurality of ablation electrodes to be activate ablation electrodes based, at least in part, on the image.

In Example 17, the ablation system of Example 16, wherein the recommendation is based, at least in part, on comparing contrast of pixels in the image around the ablation electrodes.

In Example 18 the ablation system of any of Examples 16 and 17, wherein the one or more processors is configured to determine which of the ablation electrodes are selectable based, at least in part, on the comparison of pixels in the image.

In Example 19, the ablation system of any of Examples 16-18, wherein the recommendation is also based, at least in part, on impedance measurements taken by the plurality of ablation electrodes.

In Example 20, the ablation system of any of Examples 16-19, wherein the recommended ablation electrodes are ablation electrodes associated with an impedance measurement within a predetermined range of impedance values.

In Example 21, the ablation system of any of Examples 16-20, wherein only some of the plurality of ablation electrodes are selectable, wherein the one or more processors is configured to assign each of the selected ablation electrodes to be either a sink or a source.

In Example 22, the ablation system of any of Examples 16-21, wherein the recommendation is based, at least in part, on limiting estimated power received by sink electrodes below a predetermined threshold.

In Example 23, the ablation system of any of Examples 16-22, wherein the recommended ablation electrodes form a closed circuit.

In Example 24, the ablation system of any of Examples 16-23, wherein the recommendation is based, at least in part, on images of the ablation electrodes taken over multiple cardiac cycles.

In Example 25, the ablation system of any of Examples 16-24, wherein the ablation catheter includes an expandable member carrying the plurality of ablation electrodes, wherein the camera is positioned within the expandable member.

In Example 26, a computing device for generating and using a graphical user interface (GUI) is disclosed. The computing device includes one or more integrated circuits configured to generate a graphical representation of a plurality of electrodes of an ablation catheter for displaying via the GUI and to automatically highlight a subset of the plurality of electrodes on the GUI as active electrodes based, at least in part, on impedance values associated with each of the plurality of electrodes.

In Example 27, the computing device of Example 26, wherein automatically highlighting is further based, at least in part, on analysis of images including the electrodes of the ablation catheter.

In Example 28, the computing device of Example 27, wherein the analysis includes comparing contrast of pixels in the images around the electrodes.

In Example 29, the computing device of any of Examples 27 and 28, wherein the analysis includes recognizing each of the electrodes in the images.

In Example 30, the computing device of any of Examples 27-29, wherein the analysis is carried out, at least partially, by a trained neural network.

In Example 31, the computing device of any of Examples 26-30, wherein the one or more integrated circuits is further configured to automatically designate each of the highlighted subset of the plurality of electrodes to be either a source electrode or a sink electrode.

In Example 32, the computing device of Example 31, wherein the one or more integrated circuits is further configured to automatically assign each of the source electrodes an amount of energy.

In Example 33, the computing device of any of Examples 31 and 32, wherein the assigned amount of energy is based, at least in part, on an estimated power calculated for each of the designated sink electrodes.

In Example 34, the computing device of Example 33, wherein the assigned amount of energy is based, at least in part, on balancing the calculated estimated power among the designated sink electrodes.

In Example 35, the computing device of any of Examples 26-34, wherein the automatically highlighting is further based, at least in part, determining which of the electrodes of the ablation catheter are selectable.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows an ablation system, in accordance with certain embodiments of the present disclosure.

FIG. 2 shows a perspective view of an ablation catheter, in accordance with certain embodiments of the present disclosure.

FIGS. 3 and 4 show various views of a graphical user interface, in accordance with certain embodiments of the present disclosure.

FIG. 5 shows a schematic representation of an electrode selection process, in accordance with certain embodiments of the present disclosure.

FIGS. 6A-C show images captured by a camera of the ablation catheter of FIG. 2, in accordance with certain embodiments of the present disclosure.

FIG. 7 shows a schematic representation of features of a neural network, in accordance with certain embodiments of the present disclosure.

FIG. 8 shows a block representation of steps in a method for recommending an ablation path, in accordance with certain embodiments of the present disclosure.

FIG. 9 shows a schematic representation of electrodes of the ablation catheter of FIG. 2, in accordance with certain embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Cardiac ablation is a procedure during which cardiac tissue is treated to inactivate the tissue. The tissue targeted for ablation may be associated with improper electrical activity, as described above. Cardiac ablation can create lesions in the tissue and prevent the tissue from improperly generating or conducting electrical signals. For example, lesions in the form of a line or a circle can block the propagation of errant electrical signals. Control of the shape, depth, uniformity, etc., of the lesion is desirable.

Certain embodiments of the present disclosure involve systems, devices, and methods that can be used in connection with cardiac ablation therapy via ablation electrodes on an ablation catheter. In particular, the present disclosure describes approaches for recommending and/or selecting which ablation electrodes to activate for therapy. The recommended ablation path helps augment the process of determining which electrodes to activate during ablation procedures. Further, the present disclosure describes graphical user interfaces that display and enable control of graphical representations of features of the ablation catheter and can be used for viewing, selecting, and modifying ablation parameters, among other things.

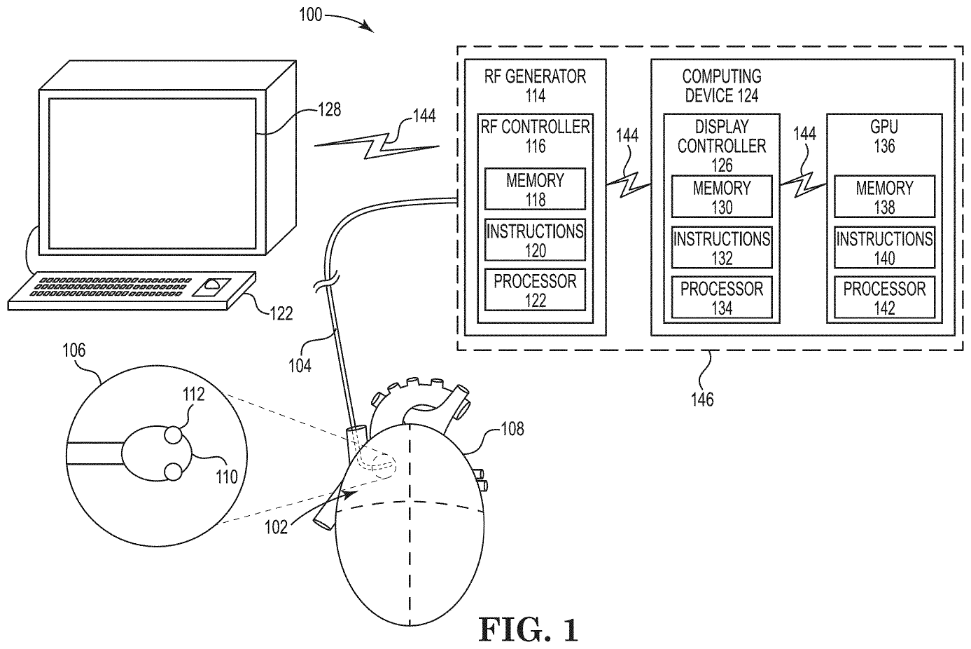

FIG. 1 shows an ablation system 100 including an ablation catheter 102 comprising an elongated catheter body 104 and a distal catheter region 106, which is configured to be positioned within a heart 108. The ablation catheter 102 includes an expandable member 110 (e.g., membrane, balloon) and a plurality of energy delivery elements 112 (e.g., ablation electrodes) secured to the expandable member 110. The energy delivery elements 112 are configured and positioned to deliver ablative energy (e.g., radiofrequency (RF) energy) to tissue when the expandable member 110 is inflated.

The system 100 includes a RF generator 114 electrically coupled to the plurality of energy delivery elements 112 and configured to generate RF energy. The RF generator 114 includes an RF generator controller 116 configured to control the RF energy to the plurality of energy delivery elements 112. The RF generator controller 116 can be implemented using firmware, integrated circuits, and/or software modules that interact with each other or are combined together. For example, the RF generator controller 116 may include memory 118 storing computer-readable instructions/code 120 for execution by a processor 122 (e.g., microprocessor) to perform aspects of embodiments discussed herein.

The system 100 can also include a computing device 124 (e.g., personal computer) with one or more controllers. Although multiple, distinct controllers are shown in FIG. 1 and described below, the functions of the various controllers can be implemented in fewer or more controllers (e.g., in multiple modules of a single controller) and/or multiple computing devices.

The computing device 124 of FIG. 1 is shown with a display controller 126, which is configured to communicate with various components of the system 100 and generate a graphical user interface (GUI) to be displayed via a display 128 (e.g., computer monitor, television, mobile device screen). The display controller 126 can be implemented using firmware, integrated circuits, and/or software modules that interact with each other or are combined together. For example, the display controller 126 may include memory 130 storing computer-readable instructions/code 132 for execution by one or more processors 134 (e.g., microprocessor) to perform aspects of embodiments of methods discussed herein.

The computing device 124 can also include a graphics processing unit (GPU) 136 configured to communicate with various components of the system 100. The GPU 136 can be implemented using firmware, integrated circuits, and/or software modules that interact with each other or are combined together. For example, the GPU 136 may include memory 138 storing computer-readable instructions/code 140 for execution by one or more processors 142 to perform aspects of embodiments of methods discussed herein. The GPU 136 can be configured to access other memory in the computing device 124.

The various components of the system 100 may be communicatively coupled to each other via communication links 144. In certain embodiments, the communication links 144 may be, or include, a wired communication link (e.g., a serial communication), a wireless communication link such as, for example, a short-range radio link, such as Bluetooth, IEEE 802.11, a proprietary wireless protocol, and/or the like. The term “communication link” may refer to an ability to communicate some type of information in at least one direction between at least two components and may be a persistent communication link, an intermittent communication link, an ad-hoc communication link, and/or the like. The communication links 144 may refer to direct communications between components and/or indirect communications that travel between components via at least one other device (e.g., a repeater, router, hub).

In embodiments, the memory 118, 130, 140 includes computer-readable storage media in the form of volatile and/or nonvolatile memory and may be removable, non-removable, or a combination thereof. Media examples include Random Access Memory (RAM), Read Only Memory (ROM), Electronically Erasable Programmable Read Only Memory (EEPROM), flash memory, and/or any other non-transitory storage medium that can be used to store information and can be accessed by a computing device. In certain embodiments, the ablation catheter 102 includes memory that stores information unique to the ablation catheter 102 (e.g., catheter ID, manufacturer). This information can be accessed and associated with data collected as part of an ablation procedure (e.g., patient data, ablation parameters).

The computer-executable instructions 120, 132, and 142 may include, for example, computer code, machine-useable instructions, and the like such as, for example, program components capable of being executed by the one or more processors 122, 134, and 142. Some or all of the functionality contemplated herein may be implemented in hardware and/or firmware.

In certain embodiments, the RF generator 114 and the computing device 124 are separate components housed in a single console 146.

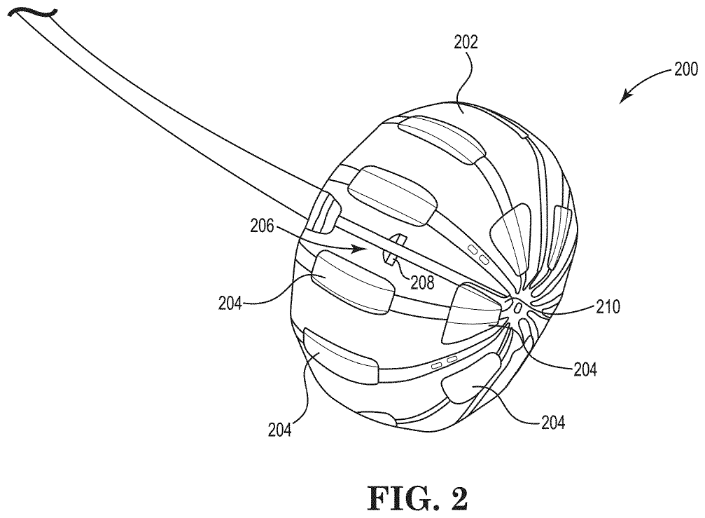

FIG. 2 shows an ablation catheter 200 that can be used in the system 100. The ablation catheter 200 includes an expandable member 202 and a plurality of energy delivery elements 204 (hereinafter referred to as ablation electrodes) secured to the expandable member 202. The ablation electrodes 204 are configured and positioned to deliver ablative energy to tissue when the expandable member 202 is inflated. As shown in FIG. 2, in certain embodiments, the ablation electrodes 204 are arranged in two rows, one proximal set of ablation electrodes 204 and one distal row of ablation electrodes 204. Each of the ablation electrodes 204 is individually addressable and/or can be used with any other ablation electrode 204. The ablation electrodes 204 can operate in a monopolar mode or bipolar mode. Sets of ablation electrodes 204 can be chosen such that the lesion is linear, a spot, a hollow circle, etc. In embodiments utilizing a monopolar mode, the system 100 may include a return pad.

The ablation catheter 200 includes a visualization system 206 including a camera assembly 208 and illumination sources (e.g., light-emitting diodes (LEDs)) disposed on a guide wire shaft 210. The camera assembly 208 can include a plurality of cameras disposed at an angle relative to a longitudinal axis of the ablation catheter 200. The cameras are configured to enable real-time imaging (e.g., video) of an ablation procedure from within the expandable member 202 including visualizing the expandable member 202, the ablation electrodes 204, and cardiac tissue as well as lesion formation during the ablation procedure. The illumination sources provide lighting for the cameras to visualize the ablation procedure. In certain embodiments, the surface of the ablation electrodes 204 facing the cameras is dark colored (e.g., painted black) to help make it easier to identify the ablation electrodes 204 in images (described in more detail below).

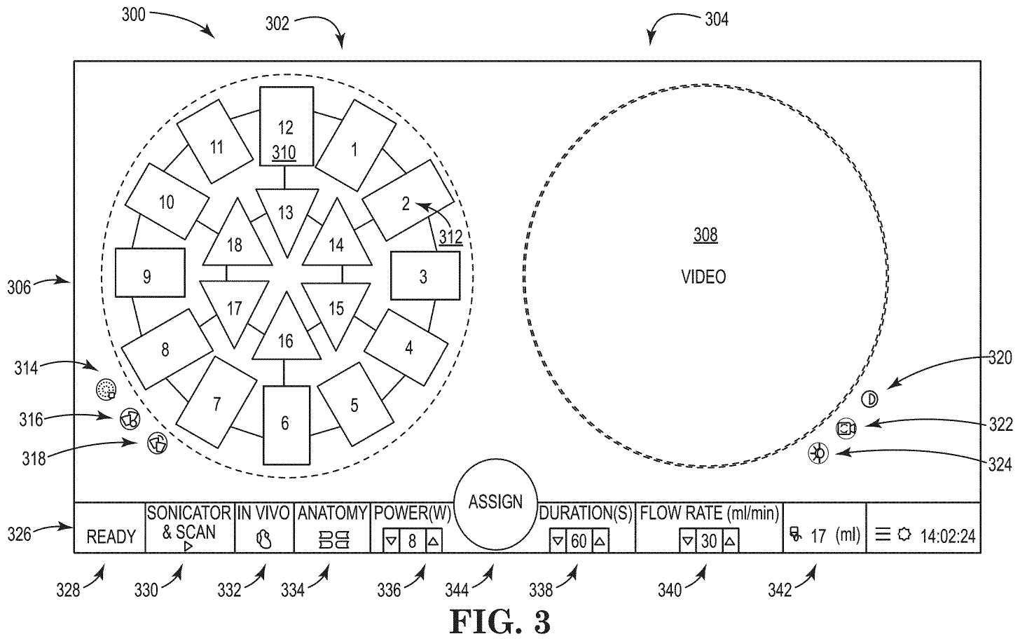

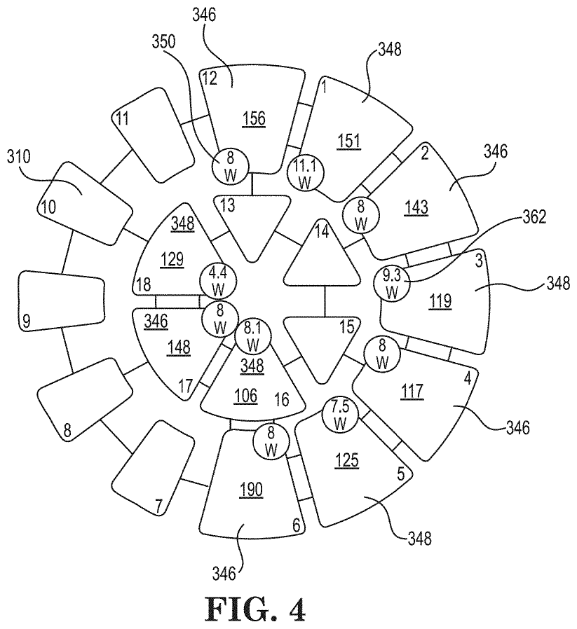

As mentioned above, the computing device 124 of the system 100 includes the display controller 126 that is configured to communicate with various components of the system 100 and generate a GUI for displaying via the display 128. FIGS. 3 and 4 show aspects of a GUI and its various features and views that can be used in the system 100 and displayed via the display 128. Users can interact (e.g., select icons, enter data) with the GUI using one or more input devices (e.g., mouse, keyboard, touchscreen). The various icons described below can take the form of selectable buttons, indicators, images, etc., on the GUI.

FIG. 3 shows a GUI 300 including a first region 302 and a second region 304. The first region 302 displays a graphical representation 306 of electrodes of an ablation catheter, and the second region 304 displays images 308 (e.g., real-time video) from the ablation catheter. The first region 302 and the second region 304 are shown as being positioned side-by-side and being circular-shaped regions. In certain embodiments, the first region 302 and the second region 304 are separate windows within the GUI 300.

The graphical representation 306 includes a separate electrode icon 310 for each of the plurality of ablation electrodes of the ablation catheter. In certain embodiments, each electrode icon 310 is similarly-shaped to an actual shape of a corresponding electrode on the ablation catheter. Each electrode icon 310 can include a unique numerical indicator 312. For example, the ablation catheter being represented by the graphical representation 306 includes twelve ablation electrodes in an outer ring and six ablation electrodes in an inner ring, and each of the electrode icons 310 is assigned an integer (e.g., 1-18). A user can select or deselect an electrode icon 310 to respectively highlight or un-highlight the electrode icon 310 on the GUI 300. As will be discussed in more detail below, in certain embodiments, the computing device 124 provides an initial recommendation of which electrodes (and therefore electrode icons 310) to activate for a desired ablation path. The initial recommendation can take the form of highlighting certain electrode icons 310 in the GUI 300.

A selected electrode icon 310 will be designated, via the GUI 300, to be an active electrode (e.g., a source electrode or a sink electrode). FIG. 4 shows an example graphical representation 306 of electrode icons 310 some of which are selected (i.e., electrodes 1-6,12, and 16-18) to be active electrodes and the rest of the electrode icons 310 unselected such that ablation electrodes associated with such electrode icons 310 will not be active during an ablation procedure.

The displayed real-time video 308 allows for visualization of an ablation procedure. The displayed real-time video 308 may include displaying video (e.g., a series of images) recorded by one or more cameras. For example, if an ablation catheter (e.g., the ablation catheter 200 of FIG. 2) includes four cameras, the real-time video 308 may display video recorded from each of the four cameras. In such embodiments, the real-time video 308 can display each of the four fields of view from the cameras overlaid with at least one other field of view.

The GUI 300 includes a number of icons (e.g., buttons, images, combinations thereof) that are associated with and can be used to control or monitor aspects of the ablation catheter and the GUI 300 itself.

FIG. 3 shows the GUI 300 including icons relating to the graphical representation 306 positioned in or near the first region 302 next to the graphical representation 306. For example, the GUI 300 includes three icons (i.e., an electrode selection icon 314, an electrode refresh icon 316, and a source-sink reverse icon 318) positioned next to the graphical representation 306 and that affect features of the ablation system 100. The electrode selection icon 314 can be used to select a pattern from a pre-determined menu of patterns of electrode icon selections 310 (e.g., inner ring of electrode icons 310, outer ring, all electrode icons 310, none). As noted above and described in more detail below, in certain embodiments, the computing device 124 provides an initial recommendation of which electrodes (and therefore electrode icons 310) to activate for a desired ablation path. Once a pattern is selected, the selected electrode icons 310 can be highlighted on the GUI 300. The electrode refresh icon 316 can be used to unselect any electrode icon 310 that has been selected. The source-sink reverse icon 318 can be used in a bipolar mode to reverse which electrode icons 310 correspond to a sink and which electrode icons 310 correspond to a source.

FIG. 3 shows the GUI 300 including icons relating to the real-time video 308 positioned in or near the second region 304 next to the real-time video 308. For example, the GUI 300 includes three icons (i.e., a contrast icon 320, a luminosity icon 322, and a video refresh icon 324) positioned next to the real-time video 308 and that affect features of the real-time video 308. The contrast icon 320 can be used to increase or decrease contrast of the real-time video 308. The luminosity icon 322 can be used to modify illumination power of illumination sources in the ablation catheter. The video refresh icon 324 can be used to refresh the video feed and/or a display controller if the real-time video 308 encounters problems.

The GUI 300 includes a ribbon 326 with various icons relating to the ablation catheter and/or the GUI 300 itself. The ribbon 326 includes a status icon 328 indicating the system's status and a sonic/scan icon 330, which initiates a routine for initiating an ultrasonic source and for scanning the electrodes on the ablation catheter to identify potentially faulty electrodes. For example, the ablation catheter may be placed in a bath coupled to an ultrasonic source, and once the sonic/scan icon 330 is selected, the routine can turn on the ultrasonic source for a predetermined period of time to remove air bubbles stuck to the ablation catheter before a treatment procedure. After expiration of the predetermined period of time, the routine can sequentially activate all electrodes to determine whether any electrodes or RF amplifiers are defective. If the ultrasonic source is not connected, the sonic/scan icon 330 will just initiate the scanning portion of the routine.

The ribbon 326 also includes an in vivo icon 332, which can be selected to indicate that the ablation catheter has been placed within a patient; an anatomy icon 334, which can be used to identify the pulmonary vein (e.g., right superior, right inferior, left superior, left inferior) to be treated; a power icon 336, which displays and allows a user to modify, via arrow buttons, a power level at which the selected ablation electrodes will be energized; a procedure timing icon 338, which displays and allows a user to modify, via arrow buttons, the length of time the selected ablation electrodes are to be energized; an irrigation flow rate icon 340, which can be used to control flow rates of irrigation fluid through the ablation catheter; and a fluid volume icon 342, which indicates the amount of fluid passed through the ablation catheter since the in vivo icon 332 was selected. Once various selections are made via the icons, data associated the selections can be stored in a memory and/or sent to a computing device (e.g., the computing device 124 of FIG. 1). For example, once a flow rate is selected, the selected flow rate can be sent to the computing device 124 to control an irrigation fluid pump.

As described above, the GUI 300 allows a user to select, via the electrode icons 310, which electrodes on the ablation catheter will be active (e.g., either a source electrode or a sink electrode). The selected and highlighted electrode icons 310 indicate that, should an ablation procedure begin, only the ablation electrodes corresponding to the selected and highlighted electrode icons 310 will be active during the ablation procedure. FIG. 3 shows the GUI 300 including an ablation activate/deactivate icon 344 with text stating “Assign” or one or more similar terms. Once the “Assign” ablation activate/deactivate icon 344 has been selected, the “Assign” text for the ablation activate/deactivate icon 344 is replaced with “Ablate”, “Start”, or one or more similar terms.

When the ablation activate/deactivate icon 344 indicates “Assign” and is selected, the computing device 124 assigns or designates the selected electrode icons 310 to be active and either a source or a sink. For example, FIG. 4 shows the selected electrode icons 310 as being either a source electrode or a sink electrode. In FIG. 4, the selected electrode icons 346 numbered “12”, “2”, “4”, “6”, and “17” are indicated as being source electrodes while the other selected electrode icons 348 (i.e., those numbered “1”, “3”, “5”, “16”, and “18”) are indicated as being sink electrodes. The unselected electrode icons in FIG. 4 (i.e., those numbered “7”, “8”, “9”, “10”, “11”, “13”, “14”, and “15”) are shown without power icons (discussed below) and without impedance values (also discussed below). In certain embodiments, the source electrodes are highlighted with a different color on the GUI 300 or are otherwise shown as being different than the sink electrodes. Further, the unselected electrode icons may be unhighlighted or faded to further visually distinguish the selected electrode icons from the unselected electrode icons.

FIG. 4 shows each of the source electrode icons 346 and the sink electrode icons 348 being associated with an impedance value in units of ohms (e.g., electrode icon “12” indicates 156 ohms, electrode icon “1” indicates 151 ohms). As will be discussed further below, the impedance values can be an input to determining a recommended ablation path.

FIG. 4 also shows each of the source electrode icons 346 and the sink electrode icons 348 including icons (e.g., power icons) indicating electrical units associated with the given electrode. For simplicity, the description below uses power in the form of Watts as the exemplary electrical unit displayed and modified via the GUI 300, but other electrical units (e.g., various forms of electrical energy) can be displayed and modified in place of power. FIG. 4 shows the source electrode icons 346 including a source power icon 350, which displays the power (e.g., 8 Watts) currently assigned to the corresponding ablation electrode. Each source power icon 350 can be selected to display a power selector icon to increase or decrease the power associated with the respective source electrode. FIG. 4 also shows each of the sink electrodes 348 including a power estimation icon 362. Each of the power estimation icons 362 displays an estimated power associated with the given sink electrode 348. The estimated power displayed in the power estimation icons 362 can be based, for example, on the power assigned to each of the source electrodes 346 and distances between the given sink electrode 348 and source electrodes 346. For example, a source electrode 346 will divide its energy among the sink electrodes 348 but more of its energy will be delivered to sink electrodes 348 positioned closer to the given source electrode 346.

As alluded to above, the computing device 124 can be configured to provide an initial recommendation of which ablation electrodes 204 (and therefore which electrode icons 310 to initially highlight) to activate for an ablation procedure. The recommended path helps augment the process of determining which electrodes to activate for a given ablation procedure. For example, recommending a path can make it easier and/or quicker for a physician to select which ablation electrodes 204 to activate for an ablation procedure.

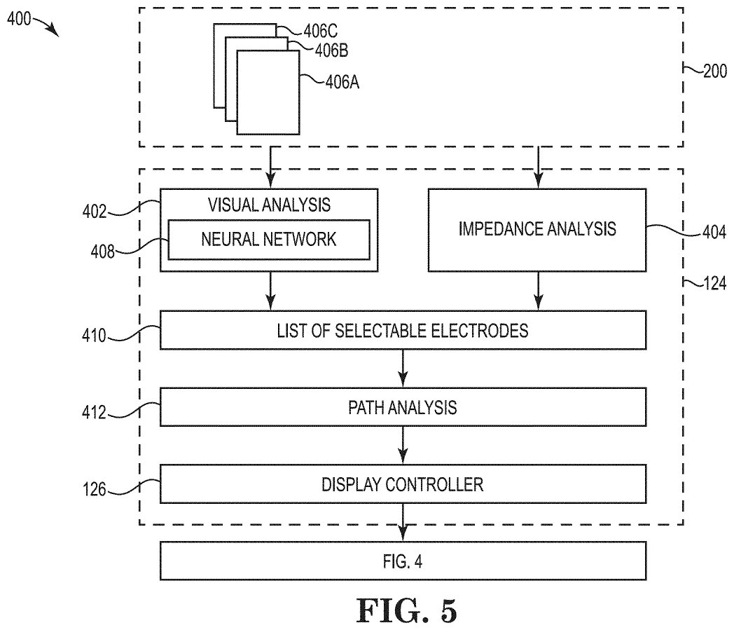

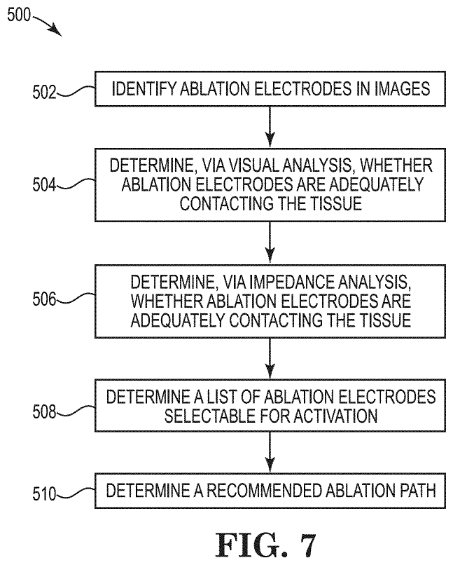

A process 400, 500 of recommending a path is shown schematically in FIG. 5 and outlined in FIG. 7. The analysis and steps shown in FIGS. 5 and 7 and described below can be carried out in parallel or sequentially in various orders. And, in certain embodiments, not all analyses and steps need to be carried out to recommend a path. The process 400, 500 involves identifying which ablation electrodes 204 are adequately contacting tissue by visual analysis 402 (e.g., analyzing images collected by one or more cameras on the ablation catheter 200) and/or impedance analysis 404 (e.g., analyzing impedance measurements of the ablation electrodes 204 on the ablation catheter 200).

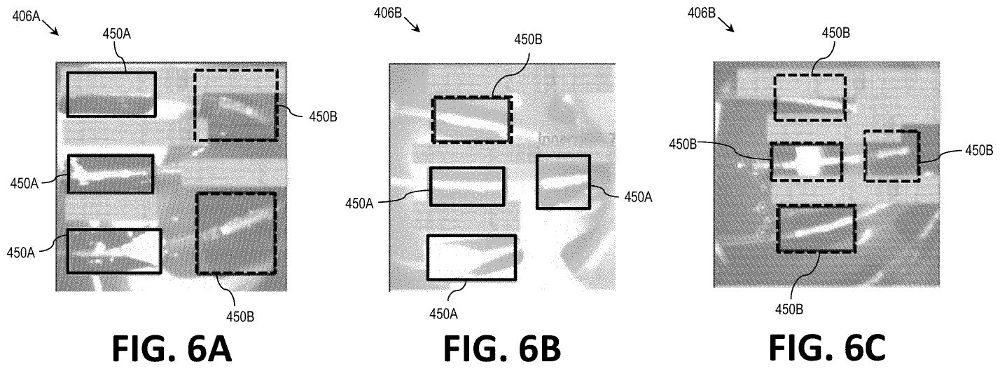

As mentioned above, the one or more cameras positioned on the ablation catheter 200 record images for display in the GUI 300. Examples images 406A-C are shown in FIGS. 6A-C. The images 406A-C, which can be taken over multiple cardiac cycles, can be inputted to the computing device 124 and analyzed to determine which of the ablation electrodes 204 are likely to be adequately contacting tissue. The visual analysis 402 can include first identifying or recognizing the ablation electrodes 204 in the images 406A-C (step 502 in FIG. 7). For example, the images 406A-C can be analyzed by a neural network 408 (described in more detail below) to determine the position and boundary of any ablation electrodes 204 in the images 406A-C. As shown in FIGS. 6A-C, each of the ablation electrodes 202 in the images 406A-C are surrounded by respective boxes 450A or 450B. These boxes 450A and 450B visually represent that the neural network 408 has identified or recognized the ablation electrodes 204 in the images 406A-C.

Once the ablation electrodes 204 in the images 406A-C are identified, the visual analysis 402 can include determining whether the identified ablation electrodes 204 are adequately contacting the tissue (step 504 in FIG. 7). In certain embodiments, the neural network 408 determines a likelihood of contact by comparing contrast of pixels in the images 406A-C around the identified ablation electrodes 204. For example, pixels around the ablation electrodes 204 that are associated with a lighter color can indicate contact with tissue while pixels that are associated with a darker color can indicate the presence of blood between one of the ablation electrodes 204 and tissue. In FIGS. 6A-6C, the boxes associated with reference number 450A are those that the neural network 408 has determined to include ablation electrodes 204 likely be in contact with tissue, while the other boxes 450B indicate ablation electrodes 204 that are not likely in contact with tissue.

In certain embodiments, the output of the visual analysis 402 is a list of ablation electrodes that are selectable for activation based on which of the ablation electrodes 204 are likely have adequate contact with tissue. In addition, each ablation electrode 204 included in the list can be associated with a level of contact or level of confidence of contact. For example, visual analysis 402 may indicate that certain ablation electrodes 204 likely have better tissue contact than other ablation electrodes 204 such as the ablation electrodes 204 surrounded by pixels with lighter color.

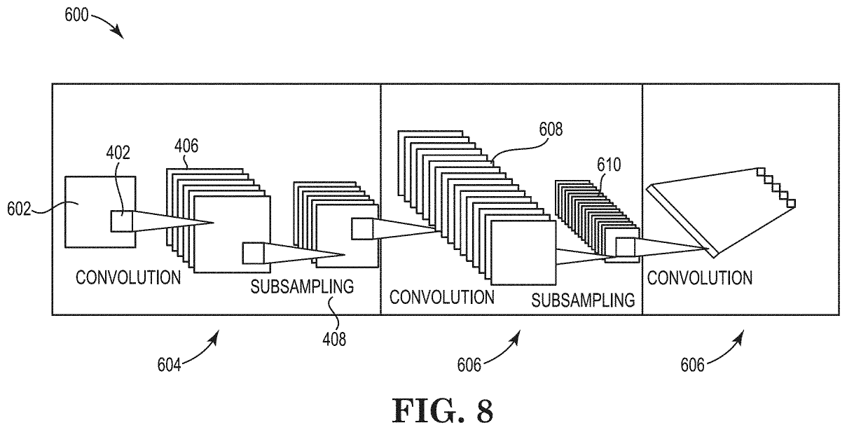

The neural network 408, generally speaking, is a computational model based on structures and functions of biological neural networks. The neural network 402 can be implemented under a variety of approaches, including a convolutional neural network (CNN) approach, among others. CCNs evaluate data (e.g., images) in the form of multiple arrays, breaking the data into a series of stages and examining the data for learned features. FIG. 8 is a simplified visual representation of an example implementation of an image analysis CNN 600. The CNN 600 (and therefore the neural network 402) can include additional features (e.g., layers).

An image 602 is inputted to the CNN 600, which abstracts the image 602 in a first convolution layer 604 to identify learned features. In a second convolution layer 606, the image 602 is transformed into a plurality of images in which the learned features are each accentuated in respective sub-images 608. The images sub-images 608 are further processed to focus on features of interest, and further processing isolates portions 510 of the images including the features of interest. The output layer 612 of the CNN 500 receives values from the last non-output layer and classifies the features of interest based on the data received from the last non-output layer.

The neural network 408 can be “trained” using supervised or unsupervised approaches. For example, a set of “training data” (e.g., known inputs and known outputs) can be used to train the neural network 408. Using a supervised approach, the input training data can be images collected from an ablation catheter that are classified with output data (e.g., the existence/boundary of ablation electrodes; an indication of whether the ablation electrodes appear to contact tissue). The known inputs and outputs are fed into an untrained neural network or a partially-trained neural network (e.g., a neural network trained to recognize objects but not necessarily ablation electrodes), which processes that data to train itself to resolve/compute results for additional sets of data with new inputs and unknown outputs. Using unsupervised approaches, the input training data can similarly be images collected from an ablation catheter which, instead of manual or semi-automatic classifying, are compared against actual lesion characteristics resulting from the active ablation electrodes in the images. As a result, under supervised or unsupervised approaches, the trained neural network can predict outputs (e.g., existence/boundary of ablation electrodes along with an indication of whether the ablation electrodes appear to contact tissue) from a set of new inputs (e.g., new images taken from an ablation catheter).

The process 400/500 further includes the impedance analysis 404 where impedance values measured by the ablation electrodes 204 are analyzed (step 506 in FIG. 7). The impedance values can be collected and inputted to the computing device 124. For example, the GUI 300 can include a button that initiates a routine that involves activating the ablation electrodes 204 for a period of time to determine the impedance values measured by each ablation electrode 204. The computing device 124 can compare the measured impedance values to a predetermined threshold range to determine which ablation electrodes 204 are likely to be adequately contacting tissue. For example, if the predetermined threshold range is 50-250 ohms, ablation electrodes 204 measuring impedance values below 50 ohms or above 250 ohms will be determined to not be adequately contacting tissue. The computing device 124 determines a list of ablation electrodes 204 that measure impedance values within the predetermined threshold range.

The computing device 124 uses the outputs of the vision-based analysis and the impedance-based analysis to determine a list 410 of ablation electrodes 204 that are selectable for activation (step 508 in FIG. 7). In certain embodiments, selectable ablation electrodes must be determined to have adequate contact with tissue under both the vision-based analysis 402 and the impedance-based analysis 404. For example, if the vision-based analysis 402 determines that a given ablation electrodes 204 is likely contacting tissue but that same ablation electrode 204 is associated with an impedance outside the predetermined threshold range, that ablation electrode 204 will be determined to be un-selectable. In certain embodiments, additional analysis can be used to generate a list of selectable ablation electrodes 204. For example, if one of the ablation electrodes 204 is determined to be defective during the sonic/scan routine, that ablation electrode 204 can be identified as an being un-selectable. The list of selectable electrodes 410 is used as an input to ablation path analysis 412, which the computing device 124 uses to determine a recommended ablation path (step 510 in FIG. 7).

Because not all theoretically-possible paths are valid or desirable paths, the computing device 124 can apply various rules and/or preferences to eliminate certain paths and/or give more or less weight to certain paths as part of determining the recommended ablation path. One example rule is that two adjacent electrodes cannot both be sources, so paths with adjacent sources are eliminated from consideration when this rule is applied. Another example rule is that sinks cannot be associated with an estimated power value above a predetermined threshold, so paths resulting in too much power at one or more of the ablation electrodes 204 are eliminated from consideration when this rule is applied. For the uneliminated paths, certain preferences can be applied to give more or less weight to paths with preferred characteristics. An example preference is that the sinks in a given path would have similar (e.g., balanced) estimated power values, so paths resulting in similar estimated power values are weighted higher than others. Another example preference is that the recommended ablation path is “closed” (i.e., forms a closed circuit with a circumferential shape) rather than “open,” so such paths are weighted higher than others. Another example preference could include weighting the visual-based analysis differently than the impedance-based analysis. As mentioned above, the visual analysis may include values indicating the estimated level of contact of each selectable ablation electrodes 204. Ablation electrodes 204 associated with higher levels of contact can be given a higher preference in the ablation path analysis 412.

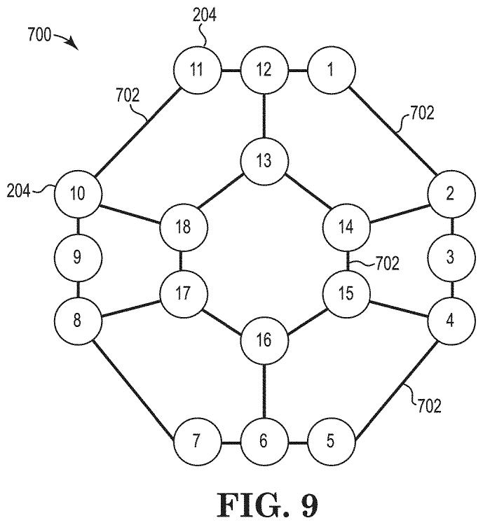

Given the constraints and preferences, the computing device 124 can determine a recommended ablation path. In certain embodiments, the computing device 124 applies graph theory approaches to determine the recommended ablation paths. When applying graph theory, the ablation electrodes can be represented as nodes (or vertices) connected by edges (e.g., undirected edges) in the model. For example, FIG. 9 shows a graph 700 of ablation electrodes 204 connected by edges 702 (only some of which are associated with reference number 702 in FIG. 9). Like the electrode icons 310 in the GUI 300, each of the ablation electrodes 204 are represented and shown to have a unique numerical identifier (e.g., “1”, “2”, through “18”). Applying graph theory, the constraints, and the preferences, the computing device 124 can determine a recommended ablation path. In certain embodiments, the computing device 124 can apply another trained neural network (e.g., using multilayer feedforward network approaches, recurrent neural network approaches) to determine a recommended ablation path.

The recommended ablation path from the ablation path analysis 412 can be communicated to the display controller 126. The display controller 126 can cause certain electrode icons 310 to become highlighted in the GUI 300 like that shown in FIG. 4. The highlighted electrode icons 310 are those associated with the ablation electrodes 204 within the recommended ablation path. In addition, the computing device 124 can automatically assign which ablation electrodes are sources and which are sinks and assign a recommended power value to the sources.

The recommended ablation path and associated parameters (e.g., sources, sinks, power) can be modified by a user via the GUI 300. Once the ablation path and associated parameters are established, the user can select the ablation activate/deactivate icon 344 (see FIG. 3). Selecting the ablation activate/deactivate icon 344 initiates and/or stops energy delivery to the ablation electrodes 204 of the ablation catheter 200. Once the activate/deactivate icon 344 is pressed to initiate energy delivery, a graphic in the activate/deactivate icon 344 changes (e.g., changes to a stop sign). Further, once the activate/deactivate icon 344 is selected to initiate energy delivery, a signal is transmitted to an RF generator (e.g., the RF generator 114 of FIG. 1) and/or an RF generator controller (e.g., the RF generator controller 116 of FIG. 1) to start delivering energy to the selected ablation electrodes 204 of the ablation catheter 200.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An ablation system comprising:
a radiofrequency (RF) generator configured to generate RF energy;
an ablation catheter in communication with the RF generator and including a plurality of ablation electrodes;
a camera positioned on the ablation catheter and arranged to take an image that includes at least some of the plurality of ablation electrodes; and
one or more processors configured to recommend a subset of the plurality of ablation electrodes to be activate ablation electrodes based, at least in part, on the image.

2. The ablation system of claim 1, wherein the recommendation is based, at least in part, on comparing contrast of pixels in the image around the ablation electrodes.

3. The ablation system of claim 2, wherein the one or more processors is configured to:
determine which of the ablation electrodes are selectable based, at least in part, on the comparison of pixels in the image.

4. The ablation system of claim 1, wherein the recommendation is also based, at least in part, on impedance measurements taken by the plurality of ablation electrodes.

5. The ablation system of claim 4, wherein recommended ablation electrodes are ablation electrodes associated with an impedance measurement within a predetermined range of impedance values.

6. The ablation system of claim 1, wherein only some of the plurality of ablation electrodes are selectable, wherein the one or more processors is configured to:
assign each of the selected ablation electrodes to be either a sink or a source.

7. The ablation system of claim 1, wherein the recommendation is based, at least in part, on limiting estimated power received by sink electrodes below a predetermined threshold.

8. The ablation system of claim 1, wherein the recommended ablation electrodes form a closed circuit.

9. The ablation system of claim 1, wherein the recommendation is based, at least in part, on images of the ablation electrodes taken over multiple cardiac cycles.

10. The ablation system of claim 1, wherein the ablation catheter includes an expandable member carrying the plurality of ablation electrodes, wherein the camera is positioned within the expandable member.

11. A computing device for generating and using a graphical user interface (GUI), the computing device comprising:
one or more integrated circuits configured to:
generate a graphical representation of a plurality of electrodes of an ablation catheter for displaying via the GUI, and
automatically highlight a subset of the plurality of electrodes on the GUI as active electrodes based, at least in part, on impedance values associated with each of the plurality of electrodes.

12. The computing device of claim 11, wherein automatically highlighting is further based, at least in part, on analysis of images including the electrodes of the ablation catheter.

13. The computing device of claim 12, wherein the analysis includes comparing contrast of pixels in the images around the electrodes.

14. The computing device of claim 12, wherein the analysis includes recognizing each of the electrodes in the images.

15. The computing device of claim 12, wherein the analysis is carried out, at least partially, by a trained neural network.

16. The computing device of claim 11, wherein the one or more integrated circuits is further configured to automatically designate each of the highlighted subset of the plurality of electrodes to be either a source electrode or a sink electrode.

17. The computing device of claim 16, wherein the one or more integrated circuits is further configured to automatically assign each of the source electrodes an amount of energy.

18. The computing device of claim 17, wherein the assigned amount of energy is based, at least in part, on an estimated power calculated for each of the designated sink electrodes.

19. The computing device of claim 18, wherein the assigned amount of energy is based, at least in part, on balancing the calculated estimated power among the designated sink electrodes.

20. The computing device of claim 11, wherein the automatically highlighting is further based, at least in part, determining which of the electrodes of the ablation catheter are selectable.
